(19) United States
(12) Patent Application Publication
(10) Pub. No.: US 2026/0363883 A1
(43) Pub. Date: Nov. 19, 2026

(54) PROSTHETIC LINER COMPRISING A GELL SLEEVE WITH A PLURALITY OF FLUID FILLED CELLS

(71) Applicant: Medico Supplies, Inc., Surrey (CA)

(72) Inventor: Terrance Bloom, Vancouver (CA)

(73) Assignee: Medico Supplies, Inc., Surrey, OT (CA)

(21) Appl. No.: 19/515,399

(22) PCT Filed: Sep. 3, 2024
(86) PCT No.: PCT/IB2024/000482

§ 371 Date: Feb. 27, 2026

Related U.S. Application Data
(60) Provisional application No. 63/580,165, filed on Sep. 1, 2023.

(51) Int. Cl. A61F 2/80; A61F 2/74; A61F 2/76; A61F 2/78

(52) U.S. Cl. CPC A61F 2/80; A61F 2/741; A61F 2002/7635; A61F 2002/7837

(57) ABSTRACT

A prosthetic liner configured to be worn over a residual limb of a user is disclosed. The disclosed prosthetic liner can include a gel sleeve configured to be worn over the residual limb and comprising a plurality of cells disposed on the gel sleeve configured to receive a fluid and exert pressure on the residual limb, a pump fluidically coupled to the gel sleeve and configured to move the fluid into the plurality of cells, and a control board electrically coupled to both the gel sleeve and the pump and configured to control activation of the pump.

### CROSS-REFERENCE TO RELATED APPLICATIONS

The present application claims priority to and the benefit of U.S. Provisional Patent Application No. 63/580,165, entitled “PROSTHETIC LINER” filed on Sep. 1, 2023, the entire teachings of which are incorporated herein by reference.

### FIELD The present disclosure generally relates to medical devices, such as prosthetic or orthopedic devices, and more particularly to a prosthetic liner used in a prosthetic device system.

### BACKGROUND

Prosthetic liners are widely known and are used as an interface between a residual limb and a prosthetic socket, allowing a user to comfortably and safely wear the prosthetic socket and prostheses attached thereto, such as prosthetic limbs. By providing a barrier between the prostheses and the residual limb, the prosthetic liners are generally designed to increase comfort for the user to wear the prostheses for long periods of time. For instance, the prosthetic liners provide cushioning between the end of the residual limb and the prosthetic socket, protecting the limb from developing pressure points as a user's weight is applied to the hard components of the prosthetic socket during regular use. The prosthetic liners may additionally provide for improved pressure distribution along the residual limb and within the prosthetic socket.

In recent times, the use of gel-based prosthetic liners have become increasingly common, with recent data suggesting that practitioners select gel-based prosthetic liners for about 85% of their prosthetic patients. As described in the above paragraph, the gel-based prosthetic liners are designed to provide comfort, protection, an intimate fit, and pressure distribution in the prosthetic fitting. The pressure distribution is achieved by the gel material flowing from areas of high pressure to areas of low pressure. To accommodate a broad range of clinical requirements prosthetic liner manufacturers have created an array of different liner products.

The mechanical properties of the gel of used in gel-based prosthetic liners are characterized as compressive elasticity (distribution of concentrated stress), shear elasticity (ability to propagate and distribute shear stresses), tensile elasticity (ability to resist axial stretching), volumetric elasticity (stiffness) and more recently thermal conductivity (ability to transfer heat away from the residual limb). More recently the term compressive elasticity has been used to describe the change in gel thickness under pressure. A prosthetic liner with a lower compressive elasticity will compress more. The clinical importance of this is that a prosthetic liner with high compressive elasticity will not distribute localized pressure well, however it will create an intimate fitting between the limb and the socket. Currently a practitioner must select from the prosthetic liner products to obtain the properties that would best serve the prosthetic user.

The prosthetic liners presently available generally work well under ideal circumstances, i.e. the prosthetic fitting system consisting of the socket/liner/limb are a contained system. However, volume management is the most common problem experienced by prosthetic liner users with lower-limb amputations. In instances when the user's limb volume changes (i.e., change in external pressure leading to change in the volume of blood in the residual limb), the volume containment is compromised, and the gel flow/pressure distribution process of the prosthetic liner is disrupted. There is a need for a prosthetic liner that can adapt to the changes in volume of the residual limb (i.e., maintain the hydrostatic principles of pressure distribution) to maintain an optimum fit when prostheses are worn.

### SUMMARY

The present disclosure relates to medical devices, such as prosthetic or orthopedic devices, and more particularly to a prosthetic liner used in a prosthetic device system.

According to one aspect of the present disclosure, there is provided a prosthetic liner configured to be worn over a residual limb of a user comprising a gel sleeve configured to be worn over the residual limb and comprising a plurality of cells disposed on the gel sleeve configured to receive a fluid and exert pressure on the residual limb, a pump fluidically coupled to the gel sleeve and configured to move the fluid into the plurality of cells, and a control board electrically coupled to both the gel sleeve and the pump and configured to control activation of the pump.

Other aspects and advantages of the invention can become apparent from the following drawings and description, all of which illustrate the various aspects of the invention, by way of example only.

### BRIEF DESCRIPTION OF THE DRAWINGS

FIG. 1A is a schematic illustration of a prosthetic fitted limb according to some aspects disclosed herein.

FIG. 1B is a schematic illustration of a prosthetic liner according to an exemplary aspect disclosed herein.

FIG. 2 illustrates a perspective view of an embodiment of a gel sleeve of the prosthetic liner according to some aspects disclosed herein.

FIG. 3 illustrates a perspective view of an embodiment of a pump of the prosthetic liner according to some aspects disclosed herein.

FIG. 4 illustrates a perspective view of an embodiment of a control board of the prosthetic liner according to some aspects disclosed herein.

FIG. 5 illustrates a perspective view of an embodiment of a bottom cap of the prosthetic liner according to some aspects disclosed herein.

### DESCRIPTION

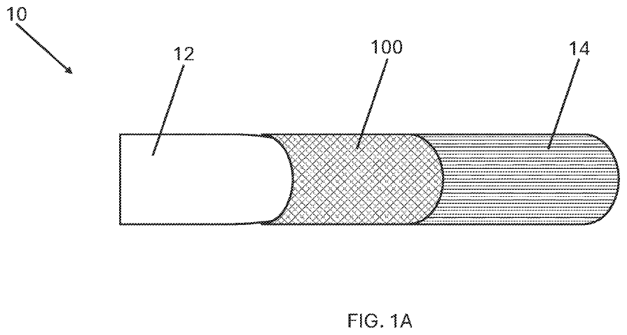

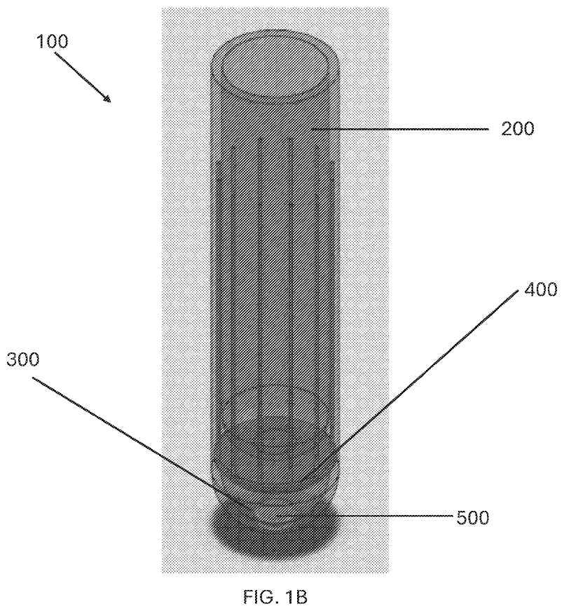

Referring to the drawings, FIG. 1A presents a schematic illustration of a prosthetic fitted limb 10 according to some aspects disclosed herein. FIG. 1A presents a prosthesis 14 fitted on to a residual limb 12. The residual limb 12 can be part of arm or leg of a user. FIG. 1A also presents an aspect of the prosthetic liner 100 which is fitted over the residual limb 12 before the prosthesis 14 is attached on the residual limb 12 covered by the prosthetic liner 100.

Referring to the drawings, FIG. 1B presents a schematic illustration of the prosthetic liner 100 according to some aspects disclosed herein. FIG. 1B also present the primary structural components of the prosthetic liner 100. These include a gel sleeve 200, a pump 300 coupled to the gel sleeve 200, a control board 400 electronically coupled to both the gel sleeve 200 and the pump 300, and bottom cap 500 coupled to the pump 300. The prosthetic liner 100 is configured to address the problem of the changing volume of the residual limb 12 to ensure comfort and optimum fit when the prosthesis 14 is worn by the user.

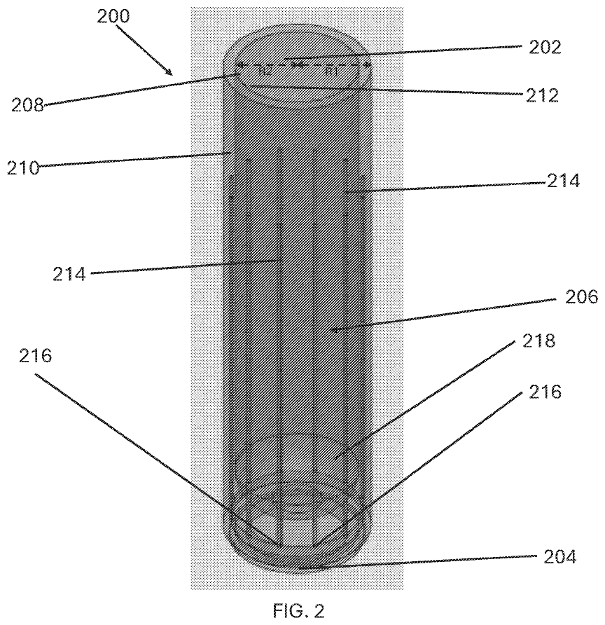

FIG. 2 illustrates a perspective view of the gel sleeve 200 of the prosthetic liner 100 according to some aspects disclosed herein. FIG. 2 also presents the primary structural components of the gel sleeve 200. These include a body 206 comprising an inner sleeve component 208 and an outer sleeve component 210, a plurality of cells 214 disposed on the inner sleeve component 208, and each of the plurality of cells 214 comprising an inlet 216 configured to receive a fluid from the pump 300. The gel sleeve 200 can be made of a flexible gel elastomer such as silicone.

As shown in in FIG. 2, the body 206 of the gel sleeve 200 comprises a proximal end 202 and a distal end 204. The proximal end 202 of the body 206 is configured to receive the residual limb 12 such that the residual limb 12 is inserted into the inner sleeve component 208 of the gel sleeve 200 towards a residual limb cup 218 contained within the inner sleeve component 208. The residual limb cup 218 can be molded to complement to and receive an end of the residual limb 12. The residual limb cup 218 can be configured to contain padding (not shown in figure) to increase the comfort of the user when the prosthetic liner 100 is fit over the residual limb 12. The residual limb cup 218 can be closed or can be a mesh structure to allow ventilation for the residual limb 12 when the prosthetic liner 100 is worn. When the prosthetic liner 100 is fitted over the residual limb 12, the residual limb 12 is configured to be rested on the residual limb cup 218 and to be in contact with an inner surface of the inner sleeve component 208.

Referring to FIG. 2, the distal end 204 of the body 206 of the gel sleeve 200 is configured to receive the control board 400 and the pump 300 as explained in the paragraphs below. The control board 400 and the pump 300 can be coupled to the distal end 204 via an adhesive such as glue. The control board 400 and the pump 300 can also be mechanically coupled to the distal end 204 via a rotational mechanism (e.g., threads disposed on the distal end 204 that complement threads of the control board 400 and the pump 300).

As shown in FIG. 2, the body 206 of the gel sleeve 200 comprises the inner sleeve component 208 and the outer sleeve component 210. A thickness of the gel sleeve 200 can be defined by the difference between a radius (R1) of the outer sleeve component 210 and a radius (R2) of the inner sleeve component 208 from the center of the gel sleeve 200. The thickness of the gel sleeve 200 can be 6 millimeters (mm). When the prosthetic liner 100 is worn over the residual limb 12, the residual limb 100 contacts the inner surface 212a of the inner sleeve component 208.

As shown in FIG. 2, the plurality of cells 214 are disposed the outer surface 212b of the inner sleeve component 208. When the prosthetic liner 100 is worn on the residual limb 12, the plurality of cells 214 are configured to receive the fluid and exert pressure on the residual limb 12, which is contacting the inner surface 212a of the inner sleeve component 208. The fluid received by the plurality of cells 214 can be oil or a similarly dense liquid. The plurality of cells 214 can be disposed around evenly around a perimeter of the outer surface 212b of the inner sleeve component 208. Each of the plurality of cells 214 can have a diameter of 2 mm.

Referring to FIG. 2, each of the plurality of cells 214 comprises an inlet 216 disposed closer to the distal end 204 of the gel sleeve 200. The inlet 216 is configured to be fluidically coupled the pump 300 to receive the fluid for exerting pressure of the residual limb 12 when the prosthetic sleeve 100 is worn over the residual limb 12. The inlet 216 can also be electrically coupled to the control board 400 for regulating when and how much fluid is received by each of the plurality of cells 214. The plurality of cells 214 can further comprise pressure sensors to measure a pressure exerted at different points on the residual limb 12 and transmit the pressure data to the control board 400 coupled to the plurality of cells 214.

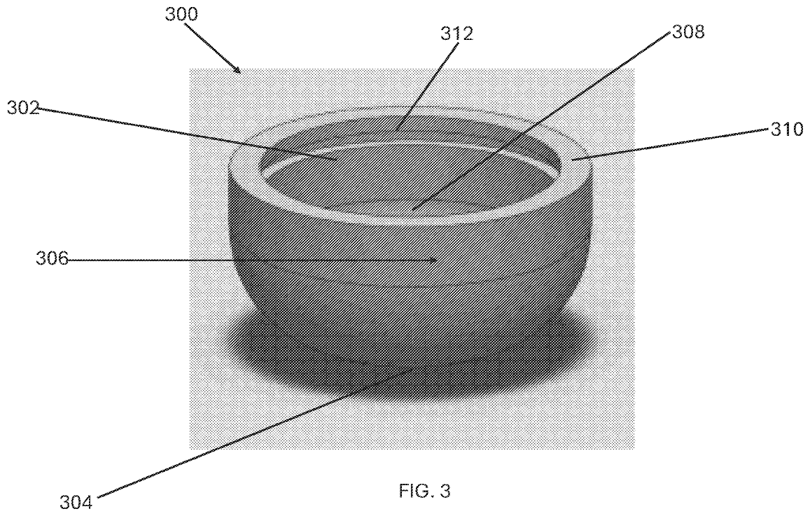

FIG. 3 illustrates a perspective view of an embodiment of the pump 300 of the prosthetic liner 100 according to some aspects disclosed herein. FIG. 3 also presents the primary structural components of the pump 300. These include a body 306 comprising a fluid reservoir 308, and a control board interface 310. The pump 300 is configured to couple to the distal end 204 of the gel sleeve 200 and pump the fluid into the plurality of cells 214 via the inlet 216. When the fluid is pumped in each of the plurality of cells 214, each of the plurality of cells 214 expand and exert pressure against the outer surface 212b of the inner sleeve component 208 and hence on the residual limb 12. Each of the plurality of cells 214 can further comprises a valve disposed at the inlet 216, the valve configured to open and allow fluid into each of the plurality of cells 214 when there is a change in pressure (i.e., change in pressure during movement of the residual limb).

As shown in in FIG. 3, the body 306 of the pump 300 comprises a proximal end 302 and a distal end 304. The proximal end 302 of the body 306 is configured to be mechanically coupled to the distal end 204 of the gel sleeve 200. In the embodiment of the pump 300 shown in FIG. 3, the proximal end 302 of the pump 300 can comprise threads 312 complementary to threads (not shown in figure) on the distal end 204 of the gel sleeve 200 such that the pump 300 can be screwed to the distal end 204 of the gel sleeve 200. The distal end 304 of the pump 300 can be configured to couple to the bottom cap 500. The distal end 304 of the pump 300 can be configured to couple to a latch (e.g., a hook or a screw) for mechanically connecting to a complementary attachment in the prosthesis 14.

As shown in FIG. 3, the body 306 of the pump 300 also comprises a fluid reservoir 308 containing a fluid. The fluid reservoir 308 is configured to be fluidically connected with the inlet 216 of each of the plurality of cells 214. In some embodiment of the pump 300, when the prosthetic liner 100 is worn over the residual limb 12, movement by the residual limb 12 can activate the pump 300 by creating a pressure gradient leading to the opening of the valve at the inlet 216 of each of the plurality of the cells 214 and produce movement of the fluid into the plurality of cells 214. In other embodiments of the pump, the pump 300 further comprises a source of power (e.g., portable battery) for electrically activating the pump 300 to move the fluid into the plurality of cells 214.

Referring to FIG. 3, the proximal end 302 of the pump 300 further comprises the control board interface 310 configured to electrically couple to the control board 400. The pump 300 can contain a source of power (e.g., battery) for activating the control board 400 via the control board interface 310. The control board 400 can also control the deactivation/activation of the pump 300 (i.e., when to pump fluid into and out of the plurality of cells 214) via the control board interface 310.

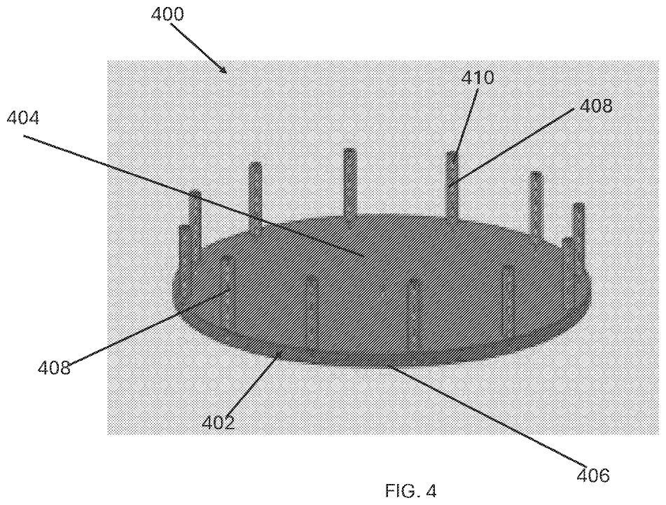

FIG. 4 illustrates a perspective view of an embodiment of a control board 400 of the prosthetic liner 100 according to some aspects disclosed herein. FIG. 4 also presents the primary structural components of the control board 400. These include a base 402 comprising a proximal surface 404 and a distal surface 406, and a plurality of controller arms 408 disposed on the proximal surface 404. The proximal surface 404 of the base 402 of the control board 400 is configured to be coupled to the distal end 204 of the gel sleeve 200. The distal surface 406 of the base 402 of the control board 400 is configured to coupled to the control board interface 310 of the pump 300.

As shown in FIG. 4, the proximal surface 404 of the base 402 comprises the plurality of controller arms 408. Each of the plurality of controller arms 408 is configured to couple to the inlet 216 of each of the plurality of the cells 214. Each of the plurality of the controller arms 408 comprises a plurality of sensors 410 (e.g., pressure sensors) to measure different variables (e.g., pressure) at different points of the residual limb 12 via the plurality of cells 214. The control board 400 is also electrically connected to the pump 300 via the control board interface 310. In response to the different variables measured by the plurality of sensors 410, the control board 400 can activate or deactivate the pump 300 to move fluid into or out of the plurality of cells 214. The control board 400 can also control the opening and closing of the valves (not shown in the figure) disposed at the inlet 216 at each of the plurality of cells 214.

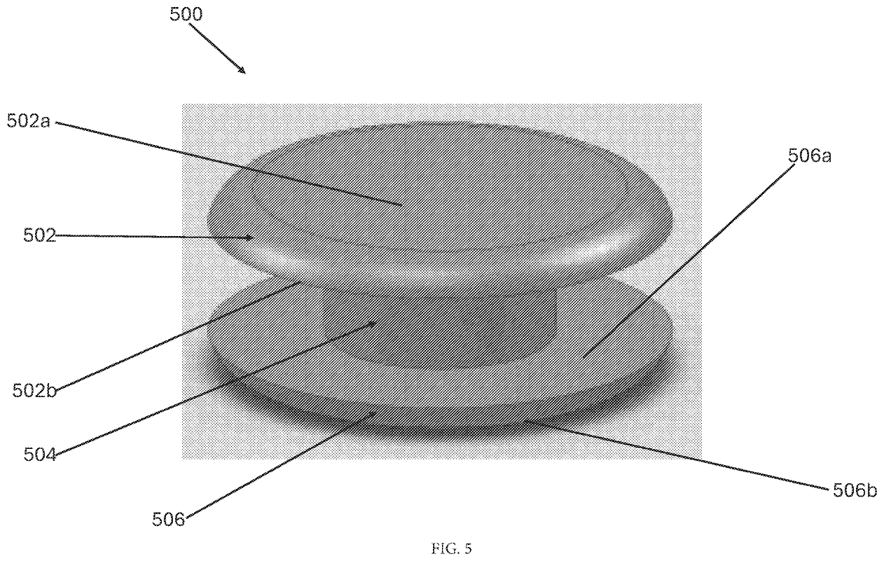

FIG. 5 illustrates a perspective view of an embodiment of a bottom cap 500 of the prosthetic liner 100 according to some aspects disclosed herein. FIG. 5 also presents the primary structural components of the bottom cap 500. These include a proximal cap component 502 with a first surface 502a and a second surface 502b, a distal cap component 506 with a first surface 506a and a second surface 506b, a body component 504 disposed between the proximal cap component 502 and the distal cap component 506. The first surface 502a of the proximal cap component 502 of the bottom cap 500 is configured to be coupled to the distal end 304 of the pump 300.

As shown in FIG. 5, the first surface 502a of the proximal cap component 502 of the bottom cap 500 is be coupled to the distal end 304 of the pump 300. The first surface 502a can be coupled to the distal end 304 of the pump 300 by an adhesive (e.g., glue) or via a rotational mechanism (e.g., threads disposed on the distal end 304 of the pump that complement threads of a screw disposed on the first surface 502a of the proximal cap component 502).

Referring to FIG. 5, the second surface 506b of the distal cap component 506 can comprise an attachment mechanism configured to secure the prosthetic liner 100 to the prosthesis 14. The attachment mechanism can be a rotational mechanism (e.g., a screw disposed on the second surface 506a of the distal cap component 506 can have threads complementary to threads disposed inside the prosthesis 14). The body component 504 of the bottom cap 500 can be configured to be height adjustable to enable the attachment mechanism of the second surface 506b of the distal cap component 506 to be coupled with different prostheses 14 with different internal depths (i.e., the complementary attachment mechanism in the prosthesis 14 being disposed at different location inside the prosthesis 14 and need the bottom cap 500 to be extended to be able to attach).

While the advantages and preferred embodiments of the present invention have been described hereinbefore, those skilled in the art should be understood that the above are merely several illustrative embodiments of the present invention without limiting the scope thereof, wherein various modifications, alterations or substitutions may be made to the specific components of the embodiments without departing from the spirit and scope of the invention and its claims.

What is claimed is:

1. A prosthetic liner configured to be worn over a residual limb of a user, the prosthetic liner comprising:
a gel sleeve configured to be worn over the residual limb, the gel sleeve comprising a plurality of cells disposed on the gel sleeve configured to:
receive a fluid; and
exert pressure on the residual limb;
a pump fluidically coupled to the gel sleeve, the pump configured to move the fluid into the plurality of cells; and
a control board electrically coupled to both the gel sleeve and the pump, the control board configured to control activation of the pump.

2. The prosthetic liner of claim 1, wherein the gel sleeve comprises a body comprising a proximal end and a distal end, wherein the proximal end is configured to receive the residual limb.

3. The prosthetic liner of claim 2, wherein the body of the gel sleeve further comprises:
an inner sleeve component comprising an inner surface and an outer surface, wherein the plurality of cells are disposed on the outer surface of the inner sleeve component; and
an outer sleeve component surrounding the inner sleeve component, wherein a thickness of the gel sleeve is a difference between a radius of the outer sleeve component and a radius of the inner sleeve component.

4. The prosthetic liner of claim 3, further comprising a residual limb cup disposed in the inner sleeve component, the residual limb configured to receive an end of the residual limb.

5. The prosthetic liner of claim 4, wherein the residual limb cup comprises padding.

6. The prosthetic liner of claim 4, wherein the residual limb cup is molded to a shape of the end of the residual limb.

7. The prosthetic liner of claim 2, wherein the thickness of the gel sleeve is 6 mm.

8. The prosthetic liner of claim 1, wherein a thickness of each of the plurality of cells is 2 mm.

9. The prosthetic liner of claim 2, wherein the pump is fluidically coupled to the distal end of the gel sleeve.

10. The prosthetic liner of claim 9, wherein the pump comprises:
a body comprising a proximal end and a distal end;
a fluid reservoir disposed within the body of the pump, the fluid reservoir configured to contain the fluid for moving into the plurality of cells; and
a control board interface disposed on the proximal end of the body, the control board interface configured to electrically couple to the control board.

11. The prosthetic liner of claim 10, wherein each of the plurality of cells comprises an inlet in fluid communication with the fluid reservoir of the pump.

12. The prosthetic liner of claim 11, wherein the inlet of each of the plurality of cells further comprises a valve configured to allow fluid from the fluid reservoir into the inlet when the pump is activated.

13. The prosthetic liner of claim 12, wherein the pump is activated by a change in pressure during the movement of the residual limb.

14. The prosthetic liner of claim 12, wherein the pump is activated electrically by the control board via the control board interface.

15. The prosthetic liner of claim 14, wherein the control board comprises:
a base comprising a proximal surface and a distal surface, wherein the control board is electrically coupled to the pump via the distal surface; and
a plurality of controller arms disposed on the proximal surface, wherein each of the plurality of controller arms are coupled to the inlet of each of the plurality of cells;

16. The prosthetic liner of claim 15, wherein each of the plurality of arms further comprises a plurality of sensors configured to measure variables at different points of the residual limb via each of the plurality of cells.

17. The prosthetic liner of claim 16, wherein the plurality of sensors comprise at least one pressure sensor configured to measure pressure at the different points of the residual limb via each of the plurality of cells.

18. The prosthetic liner of claim 10, further comprising a bottom cap disposed on the distal end of the body of the pump, the bottom cap configured to attach the prosthetic liner to the prosthetic.

19. The prosthetic liner of claim 18, wherein the bottom cap further comprises:
a proximal cap component comprising a first surface and a second surface;
a distal cap component comprising a first surface and a second surface; and
a body disposed between the proximal cap component and the distal cap component.

20. The prosthetic liner of claim 19, wherein the distal cap component further comprises an attachment mechanism configured to secure the prosthetic liner to the prosthetic.
